(19) United States
(12) Patent Application Publication
(10) Pub. No.: US 2012/0225143 A1
(43) Pub. Date: Sep. 6, 2012

(54) COLD SORE FORMULATION AND RELATED METHOD OF MANUFACTURE

(75) Inventors: Debra C. Soliz, Caledonia, MI (US); James P. Polansky, San Diego, CA (US)

(73) Assignee: RANIR, LLC, Grand Rapids, MI (US)

(21) Appl. No.: 13/039,993

(22) Filed: Mar. 3, 2011

(51) Int. Cl. A61K 38/05; A61P 31/20

(52) U.S. Cl. 424/744

(57) ABSTRACT

An composition for application to a disordered tissue, the composition including an antipathogenic agent, a healing agent and an optional soothing agent. The antipathogenic agent can include cineole, and the healing agent can include an amino acid such as L-arginine or L-carnosine. The cineole can be of dual function, for example, it can operate as an antipathogenic agent, and also operate to enhance penetration of at least one of the antipathogenic agent, healing agent and/or soothing agent into the disordered tissue, which can be skin of a subject. The disordered tissue can be afflicted by an infection, for example, a Type I herpes infection, and can be in the form of a cold sore. The composition can be in a form, such as a cream or a lip balm, adapted for topical application to the disordered tissue. A method for treating the disordered tissue with the composition is also provided.

### BACKGROUND OF THE INVENTION

The present invention relates to treatment compositions, and more particularly to treatment compositions including antipathogenic, healing and soothing agents and related methods of manufacture and use.

There are a variety of viral and bacterial infections that cause pain and discomfort for those afflicted. These types of infections can be particularly problematic where they cause topical symptoms. As an example, the herpes simplex virus (herpes) can cause blisters and sores on the skin, many times either around the mouth, nose, or generally in the facial area. Symptoms of herpes infections are frequently annoying because they may periodically reappear. Moreover, the sores caused by herpes are usually painful, unsightly and can lead to other types of health related complications.

One type of herpes is referred to as the Type 1 virus. Type 1 herpes infections many times occur in infancy or childhood. The infections can be spread by close contact with infected people, for example, through kissing or sharing food utensils. The sores usually appear shortly after exposure, and mostly affect the lips, mouth, nose, chin and/or cheeks. In less severe cases, those infected might not notice the infection or related symptoms, and may not need medication for pain relief or treating the sores.

Type 1 herpes infections can cause lesions that spread easily. For example, new herpes lesions can spread by merely touching an unaffected part of the body after touching a herpes lesion. The Type 1 lesions are typically referred to as cold sores or fever blisters. These sores usually are clear, small, fluid-filled blisters that usually develop in the facial area.

There presently are a number of medications commercially available that attempt to treat the herpes virus and its symptomatic sores or blisters. Many of them, however, are designed to quickly kill the virus, and are therefore somewhat harsh. For example, such medications can cause irritation to a treated lesion and surrounding tissue. Others medications are mostly topical, and fail to provide a mechanism to assist active agents to penetrate the skin and provide deep healing effects. Yet other medications are designed to provide a soothing effect, but fail to effectively combat and kill the virus, which leads to reduced symptoms, but does not make the infection clear up quickly. Accordingly, there remains room for improving and providing a composition that treats a herpes infection and more generally treats cold sores.

### SUMMARY OF THE INVENTION

A composition for application to a disordered tissue is provided, where the composition includes an antipathogenic agent, a healing agent and an optional soothing agent. The antipathogenic agent can include cineole, and the healing agent can include one or more amino acids, such as L-arginine and/or L-carnosine.

In one embodiment, the cineole can be of dual function, for example, it can operate as an antipathogenic agent, and also operate to enhance penetration of at least one of the antipathogenic agent, healing agent and/or soothing agent into the disordered tissue.

In another embodiment, the composition can be administered to a disordered tissue, which can be the skin of a subject. The disordered tissue can be afflicted by an infection, such as a Type I herpes infection, and can be in the form of a cold sore. The composition can be in a form, such as a cream or lip balm, adapted for topical application to the disordered tissue.

In yet another embodiment, the composition can include additional or other antipathogenic agents, such as benzalkonium chloride, tea tree oil, thymol, zinc chloride, ethanol, camphor, and/or vitamin E.

In still another embodiment, the composition can include additional or alternative healing agents such as L-carnosine, camphor and/or aloe vera.

In even another embodiment, the composition can include optional soothing agents, which can be camphor, menthol, tea tree oil and/or aloe vera.

In another further embodiment, a method is also provided including: administering to a disordered tissue, for example, an infection such as Type 1 herpes, a composition including cineole and an amino acid, such as L-arginine and/or L-carnosine; and leaving the composition in contact with the cold sore for at least 20 minutes. Optionally, the cineole serves a dual function, operating as an antipathogenic agent to treat the infection and as a skin penetration enhancer to enhance penetration of the amino acid into skin adjacent the cold sore. Further optionally, the amino acid operates as a healing agent to heal the cold sore.

The composition and method herein provide an antipathogenic agent, a healing agent and an optional soothing agent that operate synergistically to treat a disordered tissue afflicted with an infection, such as Type I herpes infection which commonly results in cold sores. The multiple agents complement each other and provide a spectrum of desired properties to eradicate the infection, while assisting in the long term healing of the tissue afflicted with the infection, and further while optionally ameliorating the pain and/or discomfort associated with the infection.

These and other objects, advantages and features of the invention will be more readily understood and appreciated by reference to the detailed description of the invention.

### DETAILED DESCRIPTION OF THE INVENTION

### I. Overview and Definitions

The current embodiment provides a composition for application to a disordered tissue, for example, a tissue infected with an infection such as a Type I herpes infection. The tissue can be in the form of external skin, and can be so disordered that a cold sore erupts on the skin. The composition includes an antipathogenic agent, a healing agent and an optional soothing agent and an optional pharmaceutical carrier. The antipathogenic agent can include cineole, and the healing agent can include one or more amino acids, such as L-arginine, L-carnosine or some other agents.

As used herein, the term “Type I herpes infection” refers to an infection of tissue typically caused by a herpes simplex virus. One type of such infection is herpes labialis, and a particular type is oro-facial herpes labialis. Type I herpes infections typically result in cold sores, also referred to as sun sores or fever blisters. Such cold sores can be, in their advanced stages, in the form of small, clear fluid filled blisters on the facial region, neck, or other regions. A cold sore infected with Type 1 herpes is simply a cold sore that was caused by or is infested with the Type I herpes.

As used herein, the term “treat”, refers to at least one of destroying, impairing, controlling, reducing, preventing, stopping replication of, neutralizing, killing, rendering inactive and/or incapacitated a pathogen, and/or treating, reducing and/or eliminating the symptoms of a pathogen, such as a virus, microbe, bacteria, prion or fungus that has affected tissue. For example, treating Type 1 herpes with a composition can include killing or neutralizing the herpes pathogen with the composition, or any of the other activities noted above. As another example, treating a cold sore can include killing the causal Type I herpes, but also can include attaining a desired medical outcome, such as control or destruction of the affected cells or pathogens, or a desired therapeutic response, without producing unacceptable toxic symptoms, allergic reactions, and/or irritation in the tissue and/or subject.

As used herein, the term “effective amount” refers to the amount, concentration, quantity or level of the active ingredients of the composition that can attain a desired medical outcome, such as control or destruction of the affected cells or pathogens or a desired therapeutic response, without producing unacceptable toxic symptoms, allergic reactions, and/or irritation in the tissue and/or subject. Particular effective amounts can vary with such factors as the condition being treated, the condition of the tissue, the patient's physical condition, the type of patient treated, the treatment duration, the ingredients employed, and any concurrent therapy. For example, administering an effective amount of the composition can include administering the amount of the composition to a disordered tissue, infected by a Type I herpes infection, so that the composition kills or inactivates the pathogen causing the infection, assists in healing the disordered tissue, and optionally provides soothing effect to the disordered tissue and surrounding tissue.

### II. The Composition

The composition of the current embodiment includes an antipathogenic agent, a healing agent and an optional soothing agent. The composition described herein is configured for topical application to treat a cold sore caused at least in part by Type I herpes, and in some minor cases, Type II herpes. Of course, if desired, the composition can be used for application to other infections related or similar to cold sores. The following is a description of the various ingredients in the composition of the current embodiment. Unless otherwise stated, the amounts of ingredients are provided in percents by weight of the composition.

A. Antipathogenic Agent

The composition includes at least one antipathogenic agent, which as used herein, refers to an agent that has at least one of antiseptic and antiviral properties, and/or that is able to destroy, impair, control, reduce, prevent, stop replication of, neutralize, kill, render inactive and/or incapacitate a pathogen, and/or treat, reduce and/or eliminate the symptoms of a pathogen, such as a virus, microbe, bacteria, prion or fungus. As an example, an antipathogenic agent can kill and/or eliminate the symptoms of an infection caused by a herpes virus, such as a Type I herpes infection.

In the composition of the current embodiment, cineole is included as an antipathogenic agent. Cineole is a cyclic ether and a monoterpenoid, and is also referred to as Eucalyptol or 1,3,3-trimethyl-2-oxabicyclo[2,2,2]octane. It generally can be derived from a variety of plants of the genuses Eucalyptus, Cinnamonium, Kaempferis, Laurus and/or Melaleuca. In the current embodiment, the cineole is derived from Eucalyptus globulus. The amount of cineole used in the current embodiment is optionally at least about 0.5% by weight, further optionally about 0.1% to about 45.0% by weight, even further optionally about 0.5% to about 7.0% by weight, and still further optionally about 0.85% by weight.

When used with the current embodiment, cineole can serve a dual function. First, it can operate as an antipathogenic agent. Second, it can act as a skin penetration enhancer for other ingredients in the composition. For example, it can promote a 10% to 90% improvement in penetration of other ingredients, in the composition including but not limited to the L-arginine and/or L-carnosine, zinc chloride, camphor, vitamin E and others, into the skin, and in particular, into the epidermis, where Type I herpes typically propogates. With this optional enhanced skin and epidermis penetration, the penetrating ingredients can treat the cold sore and the treatment site adjacent the cold sore in an effective manner.

Another antipathogenic agent that can be included in the composition is benzalkonium chloride (BZK), which is a mixture of alkylbenzyldimethylammonium chlorides of various even-numbered alkyl chain lengths, and is a nitrogenous cationic, primarily surface-acting agent belonging to the quaternary ammonium group. It is also referred to as benzyl-dimethyl-tridecyl-azanium chloride. Benzalkonium chloride is a generally rapidly acting biocidal agent with a relatively long duration of action. It is usually active against a variety of bacteria, viruses, fungi and protozoa. The amount of benzalkonium chloride used in the current embodiment is optionally at least about 0.5% by weight, further optionally about 0.02% to about 27.0% by weight, even further optionally about 0.05% to about 15.0% by weight, and still further optionally about 0.3% to about 5.0% by weight. If desired, the BZK can be provided in solution, in which case, the active amount of BZK may be reduced due to dilution. For example, in one embodiment, the BZK can be in a 50% solution with ethanol, where the solution can be present in an amount of about 0.26% by weight of the composition. Accordingly, the actual active amount of BZK can be 0.13% by weight. Of course, in different solutions with different materials, the active amount of the BZK, or any other ingredient herein, when in a solution, can vary accordingly

Another antipathogenic agent that can be included in the composition is thymol, which is a natural monoterpene phenol derivative of cymene, C₁₀H₁₄O, isomeric with carvacrol, found in oil of thyme. It is also referred to as 2-isopropyl-5-methylphenol. Thymol is a generally rapidly acting biocidal agent with a relatively long duration of action. It is usually active against a variety of bacteria and fungi. Thymol generally can be derived from a variety of plants including Thymus glandulosus, Thymus hyemalis, Thymus vulgaris, Thymus zygis, Origanum compactum, Origanum dictamnus, Origanum onites, and Origanum vulgare. The amount of thymol used in the current embodiment is optionally at least about 0.01% by weight, further optionally about 0.1% to about 50.0% by weight, even further optionally about 1.0% to about 25.0% by weight, still even further optionally about 1.5% to about 5% by weight, and still further optionally about 2.6% by weight.

Another antipathogenic agent that can be included in the composition is a zinc compound. Zinc compounds can include zinc, zinc salts, zinc hydrates and zinc oxides. Zinc salts can include inorganic and/or organic zinc salts. More specific examples of zinc compounds include zinc, zinc chloride, zinc acetate, zinc citrate, zinc sulfate, zinc nitrate, zinc carbonate, zinc benzoate, zinc gluconate and hydrates thereof. In the current embodiment, the zinc compound can be a zinc chloride, and the amount of the zinc compound used in the current embodiment is optionally at least about 0.1% by weight, further optionally about 0.1% to about 5.0% by weight, even further optionally about 0.4% to about 1.2% by weight, and still further optionally about 0.6% by weight.

One or more alcohols can be included in the composition to function as an antipathogenic agent. Suitable alcohols include methanol, ethanol, isopropanol, butanol, polyols such as glycerol, and the like. A particular alcohol used in the current embodiment is ethanol. The amount of ethanol used in the current embodiment can be undenatured ethanol at 190 proof. The alcohol, for example, ethanol, can be present optionally in at least about 1% by weight, further optionally about 1% to about 25.0% by weight, even further optionally about 2% to about 10% by weight, and still further optionally about 8.38% by weight.

Certain tocopherols and/or tocotrienols can be present in the composition to function as antipathogenic agents. For example, vitamin E, in the form of tocopherol acetate, can be present in the composition and can have a mild antipathogenic activity. The amount of tocopherols and/or tocotrienols, for example, vitamin E, used in the current embodiment is optionally at least about 0.1% by weight, further optionally about 1% to about 5.0% by weight, even further optionally about 2% to about 3% by weight, and still further optionally about 2.4% by weight.

The composition also can include certain oils, for example, tea tree oil, jojoba oil, soya oil, sesame oil, groundnut oil, sunflower oil, olive oil, palm oil, castor oil, coconut oil, canola oil, almond oil, rosemary oil, mint oil, sage oil, garlic oil, rice bran oil, grape seed oil, safflower oil, spearmint oil, rose oil, lemon oil, orange oil, peppermint oil, camphor oil, clove oil, and/or pine-needle oil. The oils can generally can act as an emulsifying agent, however, some oils can exhibit antipathogenic activity, or other activity.

The current embodiment can include an oil, for example, tea tree oil, that exhibits antipathogenic activity. The amount of the oil, for example, tea tree oil, used in the current embodiment is optionally at least about 0.1% by weight, further optionally about 1% to about 10.0% by weight, even further optionally about 0.5% to about 1.5% by weight, and still further optionally about 0.85% by weight.

When used with the current embodiment, tea tree oil can serve a dual function. First, it can operate as an antipathogenic agent as noted above. Second, it can act as a soothing agent as described below.

Another antipathogenic agent that can be included in the composition is camphor, which is a terpenoid with the chemical formula C₁₀H₁₆O, and is referred to as 1,7,7-Trimethylbicyclo[2.2.1]heptan-2-one. Camphor is a generally rapidly acting biocidal agent. Camphor generally can be derived from a variety of plants including Cinnamonium camphora, Dryobalanops aromatica, and Ocotea usambarensis. It can also be synthetically produced from oil of turpentine. The amount of camphor used in the current embodiment is optionally at least about 0.1% by weight, further optionally about 1% to about 15.0% by weight, even further optionally about 3.0% to about 10.0% by weight, and still further optionally about 5.0% by weight.

When used with the current embodiment, camphor optionally can serve multiple functions. First, it can operate as an antipathogenic agent as noted above. Second, it can act as a soothing agent as described below. Third, it can operate as a healing agent as further described below.

Other optional antipathogenic agents can include lemon myrtle, aniseed myrtle, wild Rosella, grapefruit seed extract, iodine, chlorine and vitamin C.

B. Soothing Agent

The composition optionally includes at least one soothing agent, which as used herein, refers to an agent that can reduce irritation or inflammation of tissue, or more generally can act as an anesthetic, and/or can function to reduce and/or eliminate pain, discomfort, stinging, burning, and/or itching sensations at or around a treatment site to which the agent is applied. For example, a soothing agent can reduce and/or eliminate the irritation and inflammation of tissue at or around an infection caused by a Type I herpes infection.

In the composition of the current embodiment, aloe vera can be included as a soothing agent. The aloe vera can be in the form of an extract from an Aloe vera plant. The amount of aloe vera used in the current embodiment is optionally at least about 0.1% by weight, further optionally about 1% to about 15.0% by weight, even further optionally about 2% to about 8.0% by weight, and still further optionally about 3.5% by weight.

When used with the current embodiment, aloe vera optionally can serve a dual function. First, it can operate as an soothing agent as noted above. Second, it can act as a healing agent as described below.

Another soothing agent can be camphor, present in the amounts as described above. Yet another soothing agent can be tea tree oil, present in the amounts as described above.

In the composition of the current embodiment, menthol can be included as a soothing agent. The menthol can be L-menthol, or any other desired menthol. Menthol can be derived from peppermint or other mint oils or materials. Menthol also can be referred to as 3-p-menthanol, hexahydrothymol, menthomenthol, or peppermint camphor. The amount of menthol used in the current embodiment is optionally at least about 0.1% by weight, further optionally about 0.1% to about 5.0% by weight, even further optionally about 0.8% to about 3.0% by weight, and still further optionally about 1.0% by weight.

Other optional soothing agents included in the composition can include glycerin, sorbitol, propylene glycol, allantoin, bisabolol, panthenol and the like.

C. Healing Agent

The composition includes at least one healing agent, which as used herein, refers to an agent that can improve, enhance and/or speed up skin's natural healing processes after having been exposed to a pathogen, such as a virus, bacteria, fungus, or some other irritant. For example, a healing agent can enhance and reduce time to clear-up of skin at a location of an infection caused by a herpes virus, for example, a cold sore.

In the composition of the current embodiment, amino acids, and in particular, certain α-amino acids, such as L-arginine, can be included as a healing agent. L-arginine is referred to as (S)-2-Amino-5-guanidinopentanoic acid. The amount of L-arginine used in the current embodiment is optionally at least about 0.1% by weight, further optionally about 0.1% to about 15.0% by weight, even further optionally about 0.2% to about 0.8% by weight, and still further optionally about 0.4% by weight.

Use of L-arginine in the current embodiment to assist in the treatment of a herpes infection is believed to be surprising and unexpected because much conventional literature suggests that arginine should be avoided if subjects are prone to cold sores. General guidance is that arginine aids in the growth and reproduction of the herpes virus by exacerbating the replication of the virus in an impending or active outbreak. “Natural Remedies for Herpes Simplex” by Alan R Gaby, MD; Alternative Medicine Review, 2006 June; 11(2):93-101. Counter to these teachings against using arginine, the current embodiment can include L-arginine to assist in treating infections caused by herpes with good results.

Other amino acids, such as L-carnosine and/or lysine can be used in place of, or in addition to, L-arginine, as healing agents in the same amounts as recited above in connection with L-arginine. L-carnosine is also known as (2S)-2-[(3-Amino-1-oxopropyl)amino]-3-(3H-imidazol-4-yl)propanoic acid. Lysine is also known as 2,6-diaminohexanoic acid.

Another healing agent can be camphor, present in the amounts as described above. Yet another healing agent can be aloe vera extract, present in the amounts as described above.

D. Surfactants

The current embodiment of the composition can include one or more poloxamers, which are nonionic triblock copolymers composed of a central hydrophobic chain of polyoxypropylene (poly(propylene oxide)) flanked by two hydrophilic chains of polyoxyethylene (poly(ethylene oxide)). Generally, the poloxamer can act as a non-ionic surfactant, and can increase the miscibility of the wax described below with one or more of the above antipathogenic, healing and/or soothing agents. An example of suitable poloxamers include Poloxamer 407, for example, Pluronic F-127 available from BASF of Mount Olive, N.J. Other optional surfactants include polyethylene glycols, such as PEG 8 and PEG 32.

The amount of surfactants, for example, the Poloxamer 407, used in the current embodiment is optionally at least about 10% by weight, further optionally about 10% to about 50% by weight, even further optionally about 15% to about 30% by weight, and still further optionally about 21.1% by weight.

E. Preservatives

The composition can include preservatives to preserve the composition from deterioration, such as methylparabens, ethylparabens, propylparabens, butylparabens and/or isobutylparabens. One particular paraben based preservative can include phenoxyethanol, methylparaben, ethylparaben, butylparaben and propylparaben. An example of such preservative is Paragon MEPB commercially available from McIntyre Group Ltd. of University Park, Ill.

The amount of preservatives, for example the Paragon MEPB, used in the current embodiment is optionally at least about 0.1% by weight, further optionally about 0.1% to about 5% by weight, even further optionally about 1% to about 3% by weight, and still further optionally about 1.3% by weight.

F. Base

The composition can include a base, such as a wax base, which can be in the form of a wax, or a wax mixed with one or more other ingredients. Suitable waxes can include wax esters, fatty acid esters, silicone copolyol based waxes, beeswax, lanolin wax, carnauba wax, candelilla wax, mineral waxes, paraffin, petroleum waxes and/or polyethylene. Optionally, the wax base, and the composition in general, can be free from hydrocarbon based waxes and oils.

In the wax base, the wax can be in the form of a wax mixture, where the wax is mixed with other ingredients, such as oils. Optionally, the wax base blend can be in the form of a lip balm base. One particular base suitable for use with the composition is a Kahl Wax 6370. Generally, this particular wax base includes a variety or emollients, solvents and natural mineral waxes such as hexyldecyllaurate, hexyldecanol, cetearylisononanoate, caprylate/caprate, dipropylene glycol and ozokerite, respectively. Of course, other wax bases with other sub-ingredients can be substituted for the 6370 Kahl Wax.

The amount of the base, for example a waxy base, such as a 6370 Kahl Wax in the current embodiment is optionally at least about 25% by weight, further optionally about 25% to about 75% by weight, even further optionally about 40% to about 70% by weight, and still further optionally about 51.76% by weight.

The base of the composition can dictate the form of the composition. Accordingly, the composition can be in the form of one or more of creams, tinctures, solutions, ointments, gels, emulsions, suspensions, pills, gel caps, capsules, and the like. Generally, as used herein, creams can include tinctures, ointments, gels, emulsions and suspensions primarily intended for external topical use.

The current embodiment of the composition is in the form of a readily spreadable cream that can be packaged in a squeeze tube. Alternatively, the composition can be in the form of a lip balm, in a cream form, or in a solid but spreadable stick form as desired.

Generally, the composition can include a pharmaceutically-acceptable carrier which carries the antipathogenic agent, healing agent and an optional soothing agent. As used herein, a pharmaceutically-acceptable carrier can refer to one or more compatible solid, liquid, gel, or other filler diluents, or encapsulating substances that are suitable for administration to a human or other animal. Some suitable carriers can be the base or waxy base described above, the surfactants described above, or other carriers such as such as sugars, starches, cellulose, oils, polyols such as propylene glycol, glycerine, sugar alcohols, emulsifiers, preservatives, such as those described above, wetting agents, saline, buffer solutions and combinations of the foregoing.

### III. Methods of Manufacturing the Composition

The composition can be prepared in a variety of manners, but is generally prepared by mixing certain ingredients in a sub-batch that is added to a main batch, which is selectively heated and cooled. A method of making one non-limiting exemplary composition will now be described.

In the exemplary method, the ingredients in the following Table I are mixed according to the method following the Table.

TABLE I

| | Percent by |
|---|---|
| Ingredient | Weight |
| 6370 Kahl Wax Mixture | 51.76 |
| Poloxamer 407 | 21.1 |
| Ethanol (190 proof un-denatured) | 8.38 |
| Camphor (either crystals or extract) | 5.0 |
| Aloe Vera Extract | 3.5 |
| Thymol | 2.6 |
| Vitamin E USP-tocopherol acetate | 2.4 |
| Paragon MEPB | 1.3 |
| L-Menthol | 1.0 |
| Tea Tree Oil | 0.85 |
| Cineole/Eucalyptol | 0.85 |
| Zinc Chloride | 0.6 |
| L-Arginine | 0.4 |
| Benzalkonium Chloride 50% | 0.26 |

In one step of the method, a sub batch is prepared to insure that the volatile components are already well mixed before they are added to the main batch at the end of the process, after the temperature of the main batch is reduced. To form the sub batch, the camphor ingredient is mixed with the tea tree oil, cineole, L-menthol and thymol together in a large mixing vessel. The sub batch ingredients are mixed by hand at a sufficient rate to provide a uniform mixture.

In another step of the method, a waxy base mixture is added to a large kettle that is already heated to about 120° F. to about 160° F. Thereafter, it is mixed until it is in a generally liquid form. Full vacuum is pulled on the kettle and ingredients. The vacuum is broken and the surfactant, for example, the Poloxamer 407, is added. The mixed surfactant, i.e., the poloxamer, and the waxy base are again mixed with an impeller in the kettle turning at about 40 rpms to about 150 rpms. Full vacuum is again pulled on the combined waxy base and Poloxamer, and the ingredients are agitated for about 10 minutes.

In another step, the vacuum is broken and the mixing speed is increased with the impeller rotating at about 400 to 6000 rpms. The aloe vera, vitamin E, parabens, zinc chloride and L-arginine, are added to the main batch.

In another step, a full vacuum is pulled for about 15 minutes. Agitation and mixing is slowed and the main batch is cooled to about 35° C. The vacuum is then broken. The sub batch of ingredients above is added to the main batch, along with the ethanol alcohol and benzalkonium chloride.

In a further step, a full vacuum is pulled for about 30 minutes. After the method is completed, the composition is in the form of a cream. The cream can be further processed and packaged. For example, the cream can be packaged in squeeze tubes for distribution.

### IV. Methods of Using the Composition

The composition can be used in a variety of ways. One exemplary method will now be described. In this method, the composition is used to treat a cold sore that is caused by a Type I herpes infection at a treatment site. In this method, a user administers to the cold sore the composition, which includes, for example, cineole, L-arginine and a base. Optionally, the composition can include the other additional ingredients mentioned above, for example, the ingredients noted in Table I above. The composition can be in the form of a cream, and the user can apply the cream over the surface of the cold sore and adjacent skin tissue.

The composition can be left in contact with the cold sore optionally for at least 20 minutes, further optionally, at least about 30 minutes, and even further optionally, about 30 minutes to 1 hour or longer. During this time, the composition is in contact with the cold sore and the treatment site, the various ingredients act as their designated agents. For example, the cineole can operate as a antipathogenic agent and treat the Type I herpes, for example, it can operate to kill or reduce the reproduction of the Type I herpes. Cineole can also operate as a skin penetration enhancer and enhance the penetration of the other ingredients of the composition into the skin adjacent the cold sore, which also means that it can enhance the penetration of the ingredients through the cold sore as well as the tissue adjacent the cold sore. During this increased enhanced penetration, L-arginine can better penetrate the skin and operate as a healing agent to heal the cold sore. The other agents, mentioned in the current embodiment above, act as antipathogenic agents, and sooth and heal the cold sore and the tissue surrounding the cold sore.

Optionally, the composition can be applied to the cold sore one to five times daily. These daily treatment regimens can last for 3 to 5 or 7 to 10 days, depending on the severity of the cold sore and related symptoms. After the cold sore is in remission or healed or no longer visible, administration of the composition can be terminated.

### V. Example

The following example is supplied by way of example, not by way of limitation, to illustrate a treatment regimen and the results of a treatment regimen with the noted exemplary composition.

In this example, the composition was in the form of a cream provided in a tube form. The cream composition included the ingredients in the amounts noted in Table I above.

In this example, 25 subjects were selected for study. The objective of the study was to determine if repeated use of an external analgesic test material, specifically the composition including the ingredients in the amounts noted in Table I above, induces healing of a cold sore.

The 25 subjects were cold sore sufferers who enrolled into the study within 48 hours of having a cold sore eruption. Healing was measured by a registered nurse via a grading scale outlined in the protocol on Days 0, 3, 7 and 10 in terms of: cracking, dryness, fissuring; measured cold sore size in millimeters; and noted cold sore location. The subjects completed questionnaires providing information on self-perceived relief.

The 25 user study sample size was chosen to make the study powerful enough to detect a difference in the relevant quantitative data with a 95% confidence interval. The overall power of the study is 78%, reflecting that there is a 78% probability that the study will detect a significant difference in outcome at 95% confidence.

The study found that greater than 56.5% of subjects self-reported feeling relief after just 2 uses in the first 24 hours. With 3 applications, 65.2% of users felt relief.

The subjects were asked to quantify (self-reported) their relief as either none, minimal, very much, or complete. All (100%) responded that they felt some (minimal or higher) level of relief of itching and burning symptoms, and 91.3% responded that they felt either very much or complete relief of itching and burning. All (100%) responded that they felt some (minimal or higher) level of relief of pain and redness, and 82.6% responded that they felt either very much or complete relief of pain and redness.

Additionally, the study showed an improvement in the physical properties of the cold sore. The cracking, dryness, and fissuring properties of the cold sores were reduced between 21% and 23% after 3 days of product use, and between 83% and 86% after 10 days of use. Nearly half (48%) of all subjects experienced a complete healing of their cold sore in 10 days.

As measured by a registered nurse, the size of the cold sores was reduced by 17.7% after 3 days of product use, and by 80.9% after 10 days of use.

The objective of the study was met. Repeated use of the composition induced healing of the cold sore as demonstrated by reduction in cracking, dryness, fissuring, and cold sore size.

The above and below descriptions are those of the preferred embodiments of the invention. Various alterations and changes can be made without departing from the spirit and broader aspects of the invention as defined in the appended claims, which are to be interpreted in accordance with the principles of patent law including the doctrine of equivalents. Any references to claim elements in the singular, for example, using the articles “a,” “an,” “the,” or “said,” is not to be construed as limiting the element to the singular. Any reference to claim elements as “at least one of X, Y and Z” is meant to include any one of X, Y or Z individually, and any combination of X, Y and Z, for example, X, Y, Z; X, Y; X, Z; and Y, Z.

1. A composition for treating a cold sore, the composition comprising:
an antipathogenic agent;
a healing agent including an amino acid;
a soothing agent; and
a pharmaceutically acceptable carrier.

2. The composition of claim 1 wherein the antipathogenic agent includes cineole, and the amino acid is at least one of L-arginine and L-carnosine.

3. The composition of claim 1 wherein the antipathogenic agent includes at least one of benzalkonium chloride, tea tree oil, thymol, zinc chloride and ethanol.

4. The composition of claim 3 wherein the soothing agent is at least one of aloe vera, menthol and camphor and the pharmaceutically acceptable carrier is at least one of a waxy base, a surfactant and a preservative.

5. The composition of claim 1 wherein the antipathogenic agent includes cineole present in an amount of about 0.1 to about 45.0 percent by weight of the composition, and the healing agent includes L-arginine present in an amount of about 0.1 to about 15.0 percent by weight of the composition.

6. The composition of claim 5 wherein the cineole is present in an amount of about 0.85 percent by weight of the composition, and the L-arginine is present in an amount of about 0.4 percent by weight of the composition.

7. The composition of claim 6 wherein the antipathogenic agent includes benzalkonium chloride present in an amount of about 0.13 percent by weight of the composition, tea tree oil present in an amount of about 0.85 percent by weight of the composition, thymol present in an amount of about 2.6 percent by weight of the composition, and zinc chloride present in an amount of about 0.6 percent by weight of the composition.

8. The composition of claim 7 wherein the soothing agent includes aloe vera present in an amount of about 3.5 percent by weight of the composition, menthol present in an amount of about 1.0 percent by weight of the composition, and camphor present in an amount of about 5.0 percent by weight of the composition.

9.-11. (canceled)

12. A composition for treating a cold sore comprising:
an antipathogenic agent including:
cineole present in an amount of about 0.1 to about 45.0 percent by weight of the composition,
benzalkonium chloride present in an amount of about 0.02 to about 27.0 percent by weight of the composition,
tea tree oil present in an amount of about 0.1 to about 5.0 percent by weight of the composition,
thymol present in an amount of about 0.1 to about 25.0 percent by weight of the composition
zinc chloride present in an amount of about 0.1 to about 5.0 percent by weight of the composition, and
ethanol present in an amount of about 1 to about 25 percent by weight of the composition;
a healing agent including L-arginine present in an amount of about 0.1 to about 15.0 percent by weight of the composition;
a soothing agent including:
aloe vera present in an amount of about 1 to about 15 percent by weight of the composition, and at least one of
camphor present in an amount of about 1 to about 15 percent by weight of the composition and
menthol present in an amount of about 0.1 to about 5 percent by weight of the composition; and
a base including a waxy mixture present in an amount of about 25 to about 70 percent by weight of the composition.

13. (canceled)

14. The composition of claim 13 wherein the composition is in the form of at least one of a cream and a balm that is adapted for topical application to a cold sore and to inactivate a herpes virus present in the cold sore.

15.-20. (canceled)
